SEQUENCE LISTING (17 sequences)

SEQ ID NO: 1 - protein, 22 aa, Bacillus subtilis
MLEGKVKWFNSEKGFGFIEVEG

SEQ ID NO: 2 - protein, 35 aa, Bacillus subtilis
MLEGKVKWFNSEKGFGFIEVEGQDDVFVHFSAIQG

SEQ ID NO: 3 - protein, 6 aa, Adenovirus
GAITIG

SEQ ID NO: 4 - protein, 8 aa, Adenovirus
NSGAITIG

SEQ ID NO: 5 - protein, 12 aa, Adenovirus
LSFDNSGAITIG

SEQ ID NO: 6 - protein, 25 aa, Adenovirus
AMITKLGSGLSFDNSGAITIGNKND

SEQ ID NO: 7 - protein, 41 aa, Adenovirus
PIKTKIGSGIDYNENGAMITKLGSGLSFDNSGAITIGNKND

SEQ ID NO: 8 - protein, 51 aa, Antheraea polyphemus
SYGGEGIGNVAVAGELPVAGKTAVAGRVPIIGAVGFGGPAGAAGAVSIAGR

SEQ ID NO: 9 - protein, 18 aa, Bombyx mori
  VARIANT 10: Xaa = Gly or Asp
GNLPFLGTAXVAGEFPTA

SEQ ID NO: 10 - protein, 45 aa, Dioscoreophyllum cumminsii
FREIKGYEYQLYVYASDKLFRADISEDYKTRGRKLLRFNGPVPPP

SEQ ID NO: 11 - protein, 50 aa, Dioscoreophyllum cumminsii
GEWEIIDIGPFTQNLGKFAVDEENKIGQYGRLTFNKVIRPCMKKTIYEEN

SEQ ID NO: 12 - protein, 150 aa, Escherichia coli
MKLLKVAAIAAIVFSGSALAGVVPQYGGGGNHGGGGNNSGPNSELNIYQYGGGNSALALQTDARNSDLTITQHGGGNGADVGQGSDDSSIDLTQRGFGNSATLDQWNGKNSEMTVKQFGGGNGAADQTASNSSVNVTQVGFGNNATAHQY

SEQ ID NO: 13 - protein, 152 aa, Salmonella typhimurium
MKLLKVAAFAAIVVSGSAVAGVVPQWGGGGNHNGGGNSSGPDSTLSIYQYGSANAALALQSDARKSETTITQSGYGNGADVGQGADNSTIELTQNGFRNNATIDQWNAKNSDITVGQYGGNNAALVNQTASDSSVMVRQVGFGNNAPANQYN

SEQ ID NO: 14 - protein, 685 aa, Saccharomyces cerevisiae
MSDSNQGNNQQNYQQYSQNGNQQQGNNRYQGYQAYNAQAQPAGGYYQNYQGYSGYQQGGYQQYNPDAGYQQQYNPQGGYQQYNPQGGYQQQFNPQGGRGNYKNFNYNNNLQGYQAGFQPQSQGMSLNDFQKQQKQAAPKPKKTLKLVSSSGIKLANATKKVGTKPAESDKKEEEKSAETKEPTKEPTKVEEPVKKEEKPVQTEEKTEEKSELPKVEDLKISESTHNTNNANVTSADALIKEQEEEVDDEVVNDMFGGKDHVSLIFMGHVDAGKSTMGGNLLYLTGSVDKRTIEKYEREAKDAGRQGWYLSWVMDTNKEERNDGKTIEVGKAYFETEKRRYTILDAPGHKMYVSEMIGGASQADVGVLVISARKGEYETGFERGGQTREHALLAKTQGVNKMVVVVNKMDDPTVNWSKERYDQCVSNVSNFLRAIGYNIKTDVVFMPVSGYSGANLKDHVDPKECPWYTGPTLLEYLDTMNHVDRHINAPFMLPIAAKMKDLGTIVEGKIESGHIKKGQSTLLMPNKTAVEIQNIYNETENEVDMAMCGEQVKLRIKGVEEEDISPGFVLTSPKNPIKSVTKFVAQIAIVELKSIIAAGFSCVMHVHTAIEEVHIVKLLHKLEKGTNRKSKKPPAFAKKGMKVIAVLETEAPVCVETYQDYPQLGRFTLRDQGTTIAIGKIVKIAE

SEQ ID NO: 15 - protein, 33 aa, Myoxocephalus scorpius
MNAPARAAAKTAADALAAAKKTAADAAAAAAAA

SEQ ID NO: 16 - protein, 7 aa, Podospora anserine
GNNQQNY

SEQ ID NO: 17 - protein, 42 aa, Adenovirus [sequence omitted]

(19) United States
(12) Patent Application Publication
(10) Pub. No.: US 2009/0175894 A1
(43) Pub. Date: Jul. 9, 2009

(54) METHODS AND MATERIALS FOR PRODUCING A GENERIC ANTI-AMYLOID IMMUNE RESPONSE IN MAMMALS

(76) Inventors: Todd E. Golde, Ponte Vedra Beach, FL (US); Rudi Hrncic, Jacksonville, FL (US); Pritam Das, Ponte Vedra Beach, FL (US)

Correspondence Address: FISH & RICHARDSON P.C., PO BOX 1022, MINNEAPOLIS, MN, 55440-1022, US

(21) Appl. No.: 12/090,471

(22) PCT Filed: Oct. 11, 2006
(86) PCT No.: PCT/US2006/040085

§ 371 Date: Oct. 2, 2008

Related U.S. Application Data
(60) Provisional application No. 60/727,296, filed on Oct. 17, 2005.

(51) Int. Cl. A61K 39/02; A61K 39/00; C12Q 1/02; A61K 38/16

(52) U.S. Cl. 424/190.1; 424/184.1; 424/185.1; 435/29; 514/12

(57) ABSTRACT

Compositions containing fibrillar aggregates of amyloidogenic polypeptides and adjuvants are described as well as methods of using such compositions to induce generic anti-amyloid immune responses in mammals.

### STATEMENT AS TO FEDERALLY SPONSORED RESEARCH

This invention was made with government support under AGO18454 awarded by the National Institutes of Health and National Institute on Aging. The government has certain rights in the invention.

### TECHNICAL FIELD

This document relates to methods for producing a generic anti-amyloid immune response in a mammal, and more particularly to compositions containing fibrillar aggregates of non-human amyloidogenic polypeptides and methods of using such compositions to induce generic anti-amyloid immune responses in mammals.

### BACKGROUND

There is compelling evidence that aggregation of Aβ peptides plays a causal role in the development of Alzheimer's disease (AD). A study by Schenk et al. (1999, Nature 400:173-177) focused attention on the therapeutic potential of altering Aβ deposition by inducing a humoral immune response to Aβ. Subsequent studies have shown that Aβ immunization appears to be effective in reducing amyloid deposition in multiple mouse models when mice are immunized either actively with fibrillar Aβ or passively with intact anti-Aβ antibodies. Moreover, in the apparent absence of any effect on Aβ deposition in the brain, Aβ immunization can ameliorate a cognitive deficit in reference memory that is present in certain APP transgenic mice. Thus, even when no decrease in Aβ deposits is observed, immunization may have some therapeutic effect.

Based on these data, Aβ immunotherapy is being pursued as a potential therapeutic strategy for AD. A clinical trial of active immunization with fibrillar Aβ42 was begun several years ago. This trial was halted in phase II due to a meningio-encephalitic like presentation in about 5% of individuals, which raised serious concerns about the continued clinical development of anti-Aβ immunization. See Orgogozo et al., 2003, Neurology 61:46-54. Thus, alternative immunization strategies are needed for treatment of AD.

### SUMMARY

This document is based on the discovery that amyloidogenic polypeptides that have no significant homology to any human protein or peptide, including the human Aβ peptide itself, can be used as immunogens for inducing a generic anti-amyloid response that is capable of altering amyloid deposition in Alzheimer's disease and other amyloidoisis.

In one aspect, this document features a method for inducing a generic anti-amyloid immune response in a mammal (e.g., a human). The method includes administering to the mammal an amount of a heterologous amyloidogenic polypeptide effective for producing the generic anti-amyloid immune response, and monitoring plasma or serum from the mammal for the ability to detect a different amyloid in vitro, wherein the different amyloid is formed by a polypeptide non-homologous with the heterologous amyloidogenic polypeptide. The plasma or serum can be monitored for the ability to detect at least two different amyloids in vitro. The method further can include monitoring plasma or serum from the mammal for the ability to detect amyloid deposits in human tissue.

The amyloidogenic polypeptide can be an all D-enantiomer or a mixed D and L enantiomer. The non-human amyloidogenic polypeptide can be from the shaft sequence of an adenovirus fiber polypeptide (e.g., a polypeptide having the sequence set forth in SEQ ID NO:3, SEQ ID NO:4, SEQ ID NO:5, SEQ ID NO:6, or SEQ ID NO:7). The amyloidogenic polypeptide can be selected from the group consisting of the chorion class A polypeptide (SEQ ID NO:8) and the chorion class B polypeptide (SEQ ID NO: 9). The amyloidogenic polypeptide can be a N-terminal fragment of a bacterial cold shock protein (e.g., the polypeptide set forth in SEQ ID NO: 1 or the polypeptide set forth in SEQ ID NO:2).

The amyloidogenic polypeptide can be a curlin protein having the amino acid sequence set forth in SEQ ID NO: 12, a fragment of the polypeptide set forth in SEQ ID NO: 12, or a curlin related protein such as the AgfA protein having the amino acid sequence set forth in SEQ ID NO:13. The amyloidogenic polypeptide can be a fragment of Sup35 or Ure2p from Saccyromyces. The amyloidogenic polypeptide can be an antifreeze polypeptide-3 having the amino acid sequence set forth in SEQ ID NO: 15 or a related polypeptide. The amyloidogenic polypeptide can be a fragment of HET-s protein from Podospora anserina. The amyloidogenic polypeptide can be a fungal hydrophobin, a chaplin from Streptomycetes spp., the monnelin chain A polypeptide of SEQ ID NO: 10, or the monellin chain B polypeptide of SEQ ID NO: 11. The amyloidogenic polypeptide can be FIgB, FlgC, FlgG or FliE or a fragment of FlgB, FlgC, FlgG or FliE. The amyloidogenic polypeptide can be Boc-β-Ala-mABA-Ome or Boc-γ-Abu-mABA-Ome.

In another aspect, this document features a composition that includes a fibrillar aggregate of a non-human amyloidogenic polypeptide and an adjuvant (e.g., alum). The aggregate can be an amyloid or a soluble oligomer, annular pore, or protofibril. The non-human amyloidogenic polypeptide can have the sequence set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:10, or SEQ ID NO: 11. The non-human amyloidogenic polypeptide also can be selected from the group consisting of the chorion class A polypeptide (SEQ ID NO:8) and the chorion class B polypeptide (SEQ ID NO: 9). In other embodiments, the non-human amyloidogenic polypeptide is a N-terminal fragment of a bacterial cold shock protein. The non-human amyloidogenic polypeptide can be a curlin protein having the amino acid sequence set forth in SEQ ID NO: 12 or a fragment of the polypeptide set forth in SEQ ID NO:12. The non-human amyloidogenic polypeptide can be the AgfA protein having the amino acid sequence set forth in SEQ ID NO: 13, or a fragment of Sup35 or Ure2p from Saccyromyces. The non-human amyloidogenic polypeptide can be an antifreeze polypeptide-3 set forth in SEQ ID NO: 15 or a related polypeptide, or a fragment of HET-s protein from Podospora anserina. In other embodiments, the non-human amyloidogenic polypeptide is a fungal hydrophobin or a chaplin from Streptomycetes spp. The non-human amyloidogenic polypeptide can be FlgB, FlgC, FlgG or FliE or a fragment of FlgB, FlgC, FlgG or FliE. The non-human amyloidogenic polypeptide can be an all D-enantiomer or a mixed D and L enantiomer. The non-human amyloidogenic polypeptide can be Boc-β-Ala-mABA-Ome or Boc-γ-Abu-mABA-Ome.

In another aspect, this document features a method for altering Aβ deposition in a patient. The method includes administering a composition to the patient, the composition including a non-human amyloidogenic polypeptide and an adjuvant. The method further can include monitoring plasma or serum from the patient for the ability to detect fibrillar amyloid beta polypeptide in vitro or in situ.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description and drawings, and from the claims.

### DESCRIPTION OF DRAWINGS

FIG. 1A and 1B are graphs depicting the anti-fibrillar Aβ1-42 reactivity of sera, 30 days (1A) and 60 days (1B) after immunization.

### DETAILED DESCRIPTION

In general, this document provides methods and materials for inducing a generic anti-amyloid humoral immune response in a mammal (e.g., a human) using aggregates of amyloidogenic polypeptides heterologous to the mammal. As used herein, “anti-amyloid humoral response” is an antibody response characterized by the generation of antibodies that recognize a conformational epitope characteristic found in the quaternary structure of an amyloid and/or a pre-amyloid aggregate. Such methods provide at least two advantages over current immunotherapy strategies. First, the methods provided herein avoid harmful T-cell responses in the immunized mammal as T-cells recognize linear epitopes, not secondary structures. Immunizations with a heterologous amyloid may result in a T-cell response against the peptide forming the amyloid, but will not result in a generic anti-amyloid T-cell response. Furthermore, by using amyloid polypeptides that are heterologous to the mammal (e.g., if a human is immunized, the amyloid polypeptides are not of human origin), problems associated with auto-reactive T-cells can be avoided. Second, immunization with heterologous amyloids can be more effective in generating high titer antibodies. The effectiveness of active immunization in elderly humans with Aβ or amyloids derived from other human peptides is likely to be limited by self-tolerance and the diminished immune response to vaccines seen in aging. Indeed, only 30% of those in the phase II trial reportedly developed high titer anti-Aβ antibodies.

amyloidogenic Polypeptides

The term “polypeptide” refers to a chain of amino acids at least 2 amino acids in length. Typically, suitable polypeptides are at least 6 amino acids in length (e.g., 6, 8, 10, 20, 25, 30, 35, 40, 45, 50, 75, 100, 150, 200, 250, 300, 400, 500, 600, or >650 amino acids in length). As used herein, “amyloidogenic polypeptides” are polypeptides that can form amyloids or pre-amyloid aggregates. Amyloid is an insoluble, ordered aggregate of peptides or proteins that are fibrillar in structure, and that can be detected by binding to Congo Red or a Thioflavin (e.g., Thioflavin T). See Merlini and Bellotti, 2003, N Engl. J. Med. 349:583-596; and Glenner, 1980, N Engl. J. Med. 302:1283-1292 for staining conditions for Congo Red and Thioflavins. Typically, an amyloid has a diameter of approximately 10 nm with lengths up to several micrometers. Pre-amyloid aggregates are smaller than amyloids (typically less than 200 nm in length), soluble, and structurally resemble a spherical particle, a curvilinear protofibril, or an annular pore. Atomic force microscopy can be used to determine the structure of pre-amyloid aggregates. Suitable amyloidogenic polypeptides that are 8 amino acids in length or longer have <40% (e.g., <35%) identity to any protein or peptide from the mammal to be immunized and include no more than 7 contiguous amino acids (e.g., 6 amino acids or less) of any protein or peptide encoded by the genome of the mammal to be immunized. In embodiments in which the polypeptide is less than 8 amino acids in length, overall homology can be >40% as such a polypeptide does not bind to MHC and should not induce autoreactive T-cells.

Percent identity of the amyloidogenic polypeptide amino acid sequence relative to another “target” amino acid sequence can be determined as follows. First, a target amino acid sequence can be compared and aligned to a subject amino acid sequence using the BLAST 2 Sequences (B12seq) program from the stand-alone version of BLASTZ containing BLASTN and BLASTP (e.g., version 2.0.14). The stand-alone version of BLASTZ can be obtained at www.fr.com/blast or www.ncbi.nlm.nih.gov. instructions explaining how to use BLASTZ, and specifically the B12seq program, can be found in the ‘readme’ file accompanying BLASTZ. The programs also are described in detail by Karlin et al, 1990, Proc. Natl. Acad. Sci. 87:2264; Karlin et al, 1990, Proc. Natl. Acad. Sci. 90:5873; and Altschul et al, 1997, Nucl. Acids Res. 25:3389.

B12seq performs a comparison between the subject sequence and a target sequence using either the BLASTN (used to compare nucleic acid sequences) or BLASTP (used to compare amino acid sequences) algorithm. Typically, the default parameters of a BLOSUM62 scoring matrix, gap existence cost of 11 and extension cost of 1, a word size of 3, an expect value of 10, a per residue cost of 1 and a lambda ratio of 0.85 are used when performing amino acid sequence alignments. The output file contains aligned regions of homology between the target sequence and the subject sequence. Once aligned, a length is determined by counting the number of consecutive nucleotides or amino acid residues (i.e., excluding gaps) from the target sequence that align with sequence from the subject sequence starting with any matched position and ending with any other matched position. A matched position is any position where an identical nucleotide or amino acid residue is present in both the target and subject sequence. Gaps of one or more residues can be inserted into a target or subject sequence to maximize sequence alignments between structurally conserved domains (e.g., α-helices, β-sheets, and loops).

The percent identity over a particular length is determined by counting the number of matched positions over that particular length, dividing that number by the length and multiplying the resulting value by 100. For example, if (i) a 500 amino acid target sequence is compared to a subject amino acid sequence, (ii) the B12seq program presents 200 amino acids from the target sequence aligned with a region of the subject sequence where the first and last amino acids of that 200 amino acid region are matches, and (iii) the number of matches over those 200 aligned amino acids is 180, then the 500 amino acid target sequence contains a length of 200 and a sequence identity over that length of 90% (i.e., 180÷200×100=90).

It will be appreciated that a target sequence that aligns with a subject sequence can result in many different lengths with each length having its own percent identity. It will also be appreciated that the length of a suitable amino acid sequence can depend upon the intended use. It is noted that the percent identity value can be rounded to the nearest tenth. For example, 78.11, 78.12, 78.13, and 78.14 are rounded down to 78.1, while 78.15, 78.16, 78.17, 78.18, and 78.19 are rounded up to 78.2. It is also noted that the length value will always be an integer.

Amyloidogenic polypeptides can include α-, β-, and γ-amino acids, natural and unnatural amino acids, and amino acids of both D- and L-stereochemistry, and can be synthesized on a solid phase. Stereochemistry of amino acids may be designated by preceding the name or abbreviation with the designation “D” or “d” or “L” or “l” as appropriate. In some embodiments, an amyloidogenic polypeptide contains all D-enantiomers. In other embodiments, the amyloidogenic polypeptide contains both D and L enantiomers. Amino acids with many different protecting groups (e.g., Boc) appropriate for use in the solid phase synthesis of polypeptides are commercially available.

The term “natural” or “naturally occurring” amino acid refers to one of the twenty most common occurring amino acids. Natural amino acids are referred to herein by their standard one letter abbreviation. The term “non-natural amino acid” or “non-natural” refers to any derivative of a natural amino acid including D forms, and β and γ amino acid derivatives. Examples of non-natural amino acids or amino acid derivatives that can be incorporated into an amyloidogenic polypeptide include the following (common abbreviations in parentheses): β-Alanine (β-Ala), γ-Aminobutyric Acid (GABA), 2-Aminobutyric Acid (2-Abu), α,β-Dehydro-2-aminobutyric Acid (Δ-Abu), γ-aminobutyric acid (γ-Abu), 1-Aminocyclopropane-1-carboxylic Acid (ACPC), Aminoisobutyric Acid (Aib), 2-Amino-thiazoline-4-carboxylic Acid, 5-Aminovaleric Acid (5-Ava), 6-Aminohexanoic Acid (6-Ahx), 8-Aminooctanoic Acid (8-Aoc), 11-Aminoundecanoic Acid (11-Aun), 12-Aminododecanoic Acid (12-Ado), 2-Aminobenzoic Acid (2-Abz), 3-Aminobenzoic Acid (3-Abz, also known as ineta-aminobenzoic acid (mABA)), 4-Aminobenzoic Acid (4-Abz), 4-Amino-3-hydroxy-6-methylheptanoic Acid (Statine, Sta), Aminooxyacetic Acid (Aoa), 2-Aminotetraline-2-carboxylic Acid (Atc), 4-Amino-5-cyclohexyl-3-hydroxypentanoic Acid (ACHPA), para-Aminophenylalanine (4-NH2-Phe), Biphenylalanine (Bip), para-Bromophenylalanine (4-Br-Phe), ortho-Chlorophenylalanine (2-Cl-Phe), meta-Chlorophenylalanine (3-Cl-Phe), para-Chlorophenylalanine (4-Cl-Phe), meta-Chlorotyrosine (3-Cl-Tyr), para-Benzoylphenylalanine (Bpa), tert-Butylglycine (Tle), Cyclohexylalanine (Cha), Cyclohexylglycine (Chg), 2,3-Diaminopropionic Acid (Dpr), 2,4-Diaminobutyric Acid (Dbu), 3,4-Dichlorophenylalanine (3,4-C12-Phe), 3,4-Diflurorphenylalanine (3,4-F2-Phe), 3,5-Diiodotyrosine (3,5-I2-Tyr), ortho-Fluorophenylalanine (2-F-Phe), meta-Fluorophenylalanine (3-F-Phe), para-Fluorophenylalanine (4-F-Phe), meta-fluorotyrosine (3-F-Tyr), Homoserine (Hse), Homophenylalanine (Hfe), Homotyrosine (Htyr), 5-Hydroxytryptophan (5-OH-Trp), Hydroxyproline (Hyp),para-Iodophenylalanine (4-I-Phe), 3-Iodotyrosine (3-I-Tyr), Indoline-2-carboxylic Acid (Idc), Isonipecotic Acid (Inp), meta-methyltyrosine (3-Me-Tyr), 1-Naphthylalaaine (1-Nal), 2 Naphthylalanine (2-Nal), para-Nitrophenylalanine (4-NO2-Phe), 3-Nitrotyrosine (3-NO2-Tyr), Norleucine (Nle), Norvaline (Nva), Ornithine (Orn), ortho-Phosphotyrosine (H2PO3-Tyr), Octahydroindole-2-carboxylic Acid (Oic), Penicillamine (Pen), Pentafluorophenylalanine (F5-Phe), Phenylglycine (Phg), Pipecolic Acid (Pip), Propargylglycine (Pra), Pyroglutamic Acid (pGlu), Sarcosine (Sar), Tetrahydroisoquinoline-3-carboxylic Acid (Tic), and Thiazolidine-4-carboxylic Acid (Thioproline, Th). It is noted that certain amino acids, e.g., hydroxyproline, that are classified as a non-natural amino acid herein, may be found in nature within a certain organism or a particular protein. For example, an amyloidogenic polypeptide can be Boc-β-Ala-mABA-Ome or Boc-γ-Abu-mABA-Ome., where “OMe” is O-methoxy is capping the COOH on the phenyl ring.

Polypeptides can be synthesized on a solid phase and purified to >95% purity (e.g., by high performance liquid chromatography (HPLC)). Aggregates of amyloidogenic polypeptides can be formed by incubating the purified polypeptides in buffer at 37° C. For example, 2 mg/mL of a polypeptide can be incubated in phosphate buffered saline (PBS) overnight at 37° C. with shaking. Once a visible precipitate is formed, various techniques can be used to confirm that the polypeptides are aggregated into amyloid or pre-amyloid. For example, one or more of the following can be used: thioflavin T and Congo Red binding, dynamic light scattering (DLS), size exclusion chromatography, SDS-PAGE, electron microscopy, or atomic force microscopy.

Non-limiting examples of amyloidogenic polypeptides include polypeptides from the amino terminus (residues 1-37) of bacterial cold shock proteins such as a Bacillus subtilis or Bacillus licheniformis major cold shock protein. For example, a suitable polypeptide can contain residues 1-25 of the B. subtilis and B. licheniformis major cold shock protein (MLEGKVKWFNSEKGFGFIEVEG, SEQ ID NO: 1) or can contain residues 1-35 of the B. subtilis and B. licheniformis major cold shock protein (MLEGKVKWFNSEKGFGFIEVEGQDDVFVHFSAIQG, SEQ ID NO:2).

Polypeptides from the shaft sequence of human adenovirus fiber proteins also can be used. For example, a suitable polypeptide can contain 6 (GAITIG, SEQ ID NO:3), 8 (NSGAITIG, SEQ ID NO:4), 12 (LSFDNSGAITIG, SEQ ID NO:5), 25 (AMITKLGSGLSFDNSGAITIGNKND, SEQ ID NO:6), or 41 (PIKTKIGSGIDYNENGAMITKLGSGLSFDNSGAITIGNKND, SEQ ID NO:7) amino acids from the shaft region (amino acids 356-396) of the adenovirus type 2 fiber protein.

Other suitable polypeptides can be derived from the chorion class A protein pc292 precursor from Antheraea polyphemus (e.g., a polypeptide having the sequence: SYGGEGIGNVAVAGELPVAGKTAVAGRVPIIGAVGFGGPAGAAGAVSIA GR, SEQ ID NO:8) or chorion protein from Bombyx inori (e.g., a polypeptide having the sequence: GNLPFLGTAXVAGEFPTA, SEQ ID NO:9, where X is G or D).

The monellin chain A (FREIKGYEYQLYVYASDKLFRADISEDYKTRGRKLLRFNGPVPPP, SEQ ID NO:10) and the monellin chain B (GEWEIIDIGPFTQNLGKFAVDEENKIGQYGRLTFNKVIRPCMKKTIYEEN, SEQ ID NO: 11) proteins from Dioscoreophyllum cumminisii and fragments of the monellin chain A and B proteins also are suitable.

Bacterial curlili/CSGA and related proteins, and fragments of such proteins also are useful. Non-limiting examples of such proteins include the curlin/CSGA protein from Escherichia coli (GenBank Accession No. CAA62282.1, GI:1147564, MKLLKVAAIAAIFSGSALAGVVPQYGGGGNHGGGGNNSGPNSELNIY QYGGGNSALALQTDARNSDLTITQHGGGNGADVGQGSDDSSIDLTQRG FGNSATLDQWNGKNSEMTVKQFGGGNGAADQTASNSSVNVTQVGFGN NATAHQY, SEQ ID NO: 12), a curlin subunit from E. coli (GenBank Accession No. AAA23616.1, GI:290425); CsgA protein from E. coli (GenBank Accession No. AAK53212.1, GI: 14039401); curlin-csgA protein from Enterobacter sakazakii (GenBank Accession No. CAD56678.1, GI:31790502), Citrobacter freundii (GenBank Accession No. CAD56675. 1, GI:31790498), or Citrobacter sp. Fec2 (GenBank Accession No. CAD56672.1, GI:31790494); major curlin subunit precursor from E. coli, e.g., E. coli CFT073 (GenBank Accession No. NP_{—}753219.1, GI:26247179) or E. coli K12 (GenBank Accession No. BAA35840.1, GI:1651514); major curlin subunit precursor from Salmonella enterica (GenBank Accession No. YP_{—}150943.1, GI:56413868), AgfA protein from Salmonella typhimurium (GenBank Accession No. CAA04151.1, GI:2275121, MKLLKVAAFAAIVVSGSAVAGVVPQWGGGGN-HNGGGNSSGPDSTLSIY QYGSANAALALQSDA KSETTITQSGYGNGADVGQGADNSTIELTQNGF RNNATIDQWNAKNSDITVGQYGGNNAALVNQTASDSSVMVRQVGFGN NAPANQYN, SEQ ID NO: 13); or SEF17 fimbrin protein from Salmonella enteritidis (GenBank Accession No. AAA98671.1, GI: 1293678).

The Sup35 protein from Saccharomyces cerevisiae (GenBank Accession No. NP_{—}010457.1, GI:6320377, MSDSNQGNNQQNYQQYSQNGNQQQGNNRYQGYQAYNAQAQPAGGY YQNYQGYSGYQQGGYQQYNPDAGYQQQYNPQGGYQQYNPQGGYQQ QFNPQGGRGNYKNFNYNNNLQGYQAGFQPQSQGMSLNDFQKQQKQAA PKPIKTLKLVSSSGIKLANATKKVGTKPAESDKKEEEKSAETKEPTKEPT KVEEPVKKEEKPVQTEEKTEEKSELPKVEDLKISESTHNTNNANVTSAD ALIKEQEEEVDDEVVNDMFGGKDHVSLIFMGHVDAGKSTMGGNLLYLT GSVDKRTIEKYEREAKDAGRQGWYLSWVMDTNKEERNDGKTIEVGKA YFETEKRRYTILDAPGHKMYVSEMIGGASQADVGVLVISARKGEYETGF ERGGQTREHALLAKTQGVNKMVVVVNKMDDPTVNWSKERYDQCVSN VSNFLRAIGYNIKTDVVFMPVSGYSGANLKDHVDPKECPWYTGPTLLEY LDTMNHVDRHINAPFMLPIAAKKDLGTIVEGKIESGHIKKGQSTLLMP NKTAVEIQNIYNETENEVDMAMCGEQVKLRIKGVEEEDISPGFVLTSPKN PIKSVTKFVAQIAIVELKSIIAAGFSCVMHVHTAIEEVHIVKLLHKLEKGT NRKSKKPPAFAKKGMKVIAVLETEAPVCVETYQDYPQLGRFTLRDQGT TIAIGKIVKIAE, SEQ ID NO:14) or the Ure2p protein from Saccharomyces cerevisiae (GenBank Accession No. AAM93191) can be used as amyloidogenic polypeptides as well as fragments of the Sup35 and Ure2p proteins. In addition, Sup35 and Ure2p related proteins and fragments of such proteins can be used. Suitable Sup35 related proteins include, for example, translation release factor 3 from Candida albicans (GenBank Accession No. AAB82541.1, GI:2582369); polypeptide release factor 3 from Zygosaccharomyces rouxii (GenBank Accession No. BAB 12684.2, GI: 13676384), Candida maltosa (GenBank Accession No. BAB 12681.2, GI: 13676380), or Debaryomyces hansenii (GenBank Accession No. BAB 12682.3, GI:15080702); a protein product from Candida glabrata CBS 138 (GenBanl Accession No. CAG58641.1, GI:49525028), Kluyveromyces lactis NRRL Y-1140 (GenBank Accession No. CAH00927.1, GI:49642965), or Debaryomyces hansenii CBS767 (GenBank Accession No. CAG85369.1, GI:49653030); SUP35 homolog from Zygosaccharomyces rouxii (GenBank Accession No. AAF 14007.1, GI:6478796), Kluyveromyces lactis (GenBank Accession No. AAF14003.1, GI:6478792), Kluyveromyces marxianus (GenBank Accession No. AAF14004.1 GI:6478793), Saccharomycodes ludwigii (GenBank Accession No. AAF14006.1, GI:6478795), or Pichia pastoris (GenBank Accession No. AAF14005. 1, GI:6478794); AGL145W protein from Ashbya gossypii (GenBank Accession No. AAS54346.1, GI:44985722); and EF-1 alpha-like protein factor from Pichia pinus (GenBank Accession No. CAA40231.1, GI:3236).

Alanine rich antifreeze polypeptides also can be used as amyloidogenic polypeptides. For example, antifreeze polypeptide SS-3 (GenBank Accession No. P04367, GI: 113894, MNAPARAAAK TAADALAAAK KTAADAAAAA AAA, SEQ ID NO: 15) and related polypeptides can be used. Non-limiting examples of SS-3 related polypeptides include antifreeze sculpin polypeptide (GenBank Accession No. 1Y04_A, GI:62738562); antifreeze polypeptide GS-5 (GenBank Accession No. P20421, GI:113904); longhorn sculpin skin-type antifreeze protein from Myoxocephalus octodecemspinosus (GenBankc Accession No. AAG22048.1, GI:10717168); antifreeze protein 3—winter flounder (Pseudopleuronectes americanus) (GenBank Accession No. FDFL3W, GI:72032); Afa5 antifreeze protein from tobacco (GenBank Accession No. AAB20142.1, GI:237857); Type I antifreeze protein from Prochlorococcus marinus (GenBank Accession No. CAE21324.1, GI:33640869); antifreeze protein—winter flounder (GenBank Accession No. 151125, GI:2134023, GenBank Accession No. JS0705, GI:85670, GenBank Accession No. CAA30389.1, GI:64212, GenBank Accession No. AAA49472.1, GI:213595 and GenBank Accession No. 1212275A, GI:225327); Afa3antifreeze protein from tobacco (GenBank Accession No. AAB20141.1, GI:237856); antifreeze protein SS-8—shorthorn sculpin (GenBank Accession No. A05163, GI:8561 1); Antifreeze protein A/B precursor (P04002, GI:113914); antifreeze polypeptide GS-8 from Myoxocephalus aenaeus (GenBank Accession No. P20617, GI: 113909); synthetic flounder antifreeze protein (GenBank Accession No. AAA72967.1, GI:554531); chain B antifreeze protein from winter flounder (GenBank Accession No. 1 WFB_B GI:1065084); skin-type antifreeze polypeptide AFP-2 from Myoxocephalus scorpius (GenBank Accession No. AAG25982.1, GI:10998655); membrane spanning protein from Shigella flexneri (GenBank Accession No. NP_{—}706495.1, GI:24111985), E. coli O157:H7 EDL933 (GenBank Accession No. AAG55075.1, GI:12513672), or E. coli K12 (GenBank Accession No. NP_{—}415267.1, GI:16128714); antifreeze prepropeptide from winter flounder (GenBank Accession No. AAB59964.1, GI:457351); putative secreted protein from Streptomyces coelicolor (GenBank( Accession No. CAB36606.1, GI:4455743 or GenBanlk Accession No. CAB62715.1, GI:6562784); COG3144, flagellar hook-length control protein from Burkholderia fungorum (GenBank Accession No. ZP_{—}00278986.1, GI:48782457); CG16779-PA (GenBank Accession No. AAF54383.1, GI:7299186) or CG7434-PA (GenBank Accession No. NP_{—}477134.1, GI: 17137152) from Drosophila melanogaster; ribosomal protein L22 from Drosophila melanogaster (GenBank Accession No. AAD19341.1, GI:4378008); Flag-tag_beta_lactamase_tolA fusion protein (GenBank Accession No. AAQ93652.1, GI:37575400); antifreeze protein AFP homolog (GenBank Accession No. AAC60714.1, GI:560670); transcriptional activator from Cryptococcus neoformans (GenBank Accession No. AAW40728.1, GI:57222684); TolA protein from E. coli CFT073 (GenBank Accession No. NP_752748.1, GI:26246708); tol protein from Salmonella typhimurium LT2 (GenBank Accession No. AAL19691.1, GI: 16419257), Mapkap1 protein from Mus musculus (GenBank Accession No. AAH48870.1, GI:28981397); protein associated to the polyhydroxyalkanoate inclusion from Pseudomonas sp. 61-3 (GenBank Accession No. BAB91367.1, GI:20502373); CG11203-PA from Drosophila melanogaster (GenBank Accession No. NP_572666.1, GI:24641144); a predicted protein from Magnaporthe grisea 70-15 (GenBank Accession No. EAA50560. 1, GI:38103924); ENSANGP00000012554 from Anopheles gambiae str. PEST (GenBank Accession No. EAA11004.2 GI:30175902); Om(1D) from Drosophila ananassae (GenBank Accession No. CAA40011.1, GI:7147); BarH1 from Drosophila ananassae (GenBank Accession No. AAA28381.1, GI: 156976); polyhydroxyalkanoate granule-associated protein PhaF from Pseudomonas syringae pv. tomato str. DC3000 (GenBank Accession No. NP_794878.1, GI:28872259); chain B reverse gyrase from Archaeoglobus fulgidus (GenBank Accession No. IGKU_B, GI:20149845); protein product from Kluyveromyces lactis (GenBank Accession No. CAG99118.1, GI:49643166), Tetraodon nigroviridis (GenBank Accession No. CAF91831.1 GI:47213557), or Limanda ferruginea (GenBank Accession No. CAA29655. 1, GI:64042); SD05989p from Drosophila melanogaster (GenBank Accession No. AAM52764.1, GI:21483578); CG7518-PB, isoform A (GenBank Accession No. AAF54888.2, GI:10726500) and isoform B (GenBank Accession No. AAN14338.1, GI:23175967) from Drosophila melanogaster; CG5529-PA from Drosophila melanogaster (GenBank Accession No. NP_{—}523387.1, GI:17737357); radial spoke protein 2 from Chlamydomnonas reinhardtii (GenBank Accession No. AAQ92371.1, GI:37528882); OmpA/MotB domain from Rhodopseudomonas palustris (GenBank Accession No. NP_{—}947119.1 GI:39934843), polyhydroxyalkanoate synthesis protein PhaF from Pseudomonas aeruginosa (GenBank Accession No. NP_{—}253747.1, GI: 15600253); exodeoxyribonuclease V, predicted protein from Gallus gallus (GenBank Accession No. XP_{—}424728.1, GI:50761474); and COG2913, small protein A (tmRNA-binding) protein from Burkholderia cepacia (GenBank Accession No. ZP_{—}00216624.1, GI:46316044). Other suitable SS-3 related polypeptides include the following hypothetical proteins: BPSS2166 from Burkholderia pseudomallei (GenBank Accession No. YP_{—}112167.1, GI:53723182), Rsph03002275 from Rhodobacter sphaeroides (GenBank Accession No. ZP_{—}00006323.2, GI:46192645), Mdeg02001428 from Microbulbifer degradans (GenBank Accession No. ZP_{—}00317244.1, GI:48863350), VNG0441H from Halobacterium sp. NRC-1 (GenBank Accession No. NP_{—}279507.1, GI: 15789683), XP_{—}579923 from Rattus norvegicus (GenBank Accession No. XP_{—}579923.1, GI:62640396), hypothetical protein from Streptomyces coelicolor A3(2) (GenBank Accession No. CAA19786.1, GI:3288614), surface protein from Bacteroides thetaiotaomicron VPI-5482 (GenBank Accession No. AAO76619.1, GI:29338820), RPA4347 from Rhodopseudomonas palustris CGA009 (GenBank Accession No. NP_{—}949683.1 GI:39937407), UM03989.1 from Ustilago maydis (GenBank Accession No. EAK84999.1, GI:46099766), hypothetical protein 4 (phaC2 3′ region) from Pseudomnonas aeruginosaor (GenBank Accession No. S29309, GI:485464), CNBH0920 from Cryptococcus neoformans (GenBank Accession No. EAL19399.1, GI:50256676), gp58 from Burkholderia cenocepacia phage BcepB1A (GenBank Accession No. YP_{—}024894.1 GI:48697536), and Oryza sativa (japonica cultivar-group) (GenBank Accession No. BAD61824.1, GI:54291151).

Other suitable polypeptides include fragments of the HET-s protein from Podospora anserine such as GNNQQNY (SEQ ID NO:16) or a fungal hydrophobin polypeptide (e.g., RodA from Aspergillus niger, GenBank Accession No. AAX21520, GI 60476801; Q9UVI4, a trihydrophobin precursor from Claviceps fusiformis, GenBank Accession No. Q9UVI4, GI:25091421; hydrophobin 3 precursor from Agaricus bisporus, GenBank Accession No. O13300, GI 12643535; hydrophobin II precursor from Hypocrea jecorina (GenBank Accession No. P79073, GI 6647555), Pisolithus tinctorius (GenBank Accession No. P52749, GI: 1708380), or Agaricus bisporus (GenBank Accession No. P49073, GI 1708379); hydrophobin-like protein ssgA precursor from Metarhizium anisopliae, GenBank Accession No. P52752, GI 1711536; hydrophobin-like protein MPG1 precursor from Magnaporthe grisea, GenBank Accession No. P52751, GI 1709085; hydrophobin I precursor from Hypocrea jecorina (GenBank Accession No. P52754; GI 1708378) or Pisolithus tinctorius (GenBank Accession No. P52748, GI 1708377); spore-wall fungal hydrophobin dewA precursor from Emericella nidulans, GenBank Accession No. P52750, GI 1706367; cryparin precursor from Cryphonectria parasitica, GenBank Accession No. P52753, GI 1706154; hydrophobin 1 from Heterobasidion annosum, GenBank Accession No. ABA46363, GI 76563862; hydrophobin 2 from Heterobasidion annosum, GenBank Accession No. ABA46362, GI 76563860; UM05010.1, a hypothetical protein from Ustilago maydis (GenBank Accession No. XP_{—}761157, GI 71021853) or Caenorhabditis elegans (GenBank Accession No. AAA81483, GI 29570473); rodlet protein precursor from Aspergillus nidulans, GenBank Accession No. XP_{—}682072, GI 67903632; spore-wall hydrophobin precursor from Aspergillus nidulans, GenBank Accession No. XP_{—}681275, GI 67902038; hydrophobin precursor from Neurospora crassa, GenBank Accession No. Q04571, GI 416771; or magnaporin from Magnaporthe grisea, GenBank Accession No. AAD18059, GI 4337063). Other examples of useful polypeptides include a chaplin from Streptomycetes spp. and related polypeptides (e.g., a small membrane protein from Streptomyces coelicolor (GenBank Accession No. NP_{—}625950.1, GI:21220171, or Accession No. NP_{—}626950, GI 21221171) or Thermobifida fusca (GenBank Accession No. YP_{—}290942, GI 72163285); a secreted protein from Streptomyces avermitilis (GenBank Accession No. NP₁₃ 827811.1, GI:29833177), Streptomyces coelicolor (GenBank Accession No. NP_{—}625949.1, GI:21220170; NP_{—}733581, GI 32141179; AAM78434, GI 21902161, NP_{—}626070, GI 21220291; or NP_{—}631313, GI 21225534); or Streptomyces avermitilis (GenBank Accession No. NP_{—}827812, GI29833178;NP 822405, GI29827771; or NP 827654, GI 29833020); a membrane protein from Streptomyces coelicolor, GenBank Accession No. NP_{—}626939, GI 21221160; or a protein from Streptomyces verticillus, GenBank Accession No. AAG43514, GI 12003276. Flagellar basal body protein from Salmonella such as FlgB, FlgC, FlgG, and FliE (GenBank Accession Nos. BAA21014, YP_{—}150913, P16323, and P26462, respectively) or fragments of such flagellar basal body proteins also are useful.

### Compositions Containing Amyloidogenic Polypeptides

In some embodiments, an aggregate of an amyloidogenic polypeptide is combined with an adjuvant to form a composition that elicits a generic anti-amyloid immune response when administered to a mammal. In other embodiments, the composition contains aggregates of two or more different amyloidogenic polypeptides. An “adjuvant” is an immunological compound that can enhance an immune response against a particular antigen such as a polypeptide. Suitable adjuvants include alum as well as other aluminum-based compounds (e.g., Al₂O₃) that can be obtained from various commercial suppliers. For example, REHYDRAGEL® adjuvants can be obtained from Reheis Inc. (Berkeley Heights, N.J.). REHYDRAGEL® adjuvants are based on crystalline aluminum oxyhydroxide, and are hydrated gels containing crystalline particles with a large surface area (about 525 m²/g). Their Al₂O₃ content typically ranges from about 2 percent to about 10 percent. Rehydragel LG, for example, has an Al₂O₃ content of about 6 percent, and flows readily upon slight agitation. Rehydragel LG also has a protein binding capacity of 1.58 (i.e., 1.58 mg of bovine serum albumin bound per 1 mg of Al₂O₃), a sodium content of 0.02 percent, a chloride content of 0.28 percent, undetectable sulphate, an arsenic level less than 3 ppm, a heavy metal content less than 15 ppm, a pH of 6.5, and a viscosity of 1090 cp. Rehydragel LG can be combined with a polypeptide solution (e.g., a polypeptide in PBS) to yield Al(OH)₃. In addition, ALHYDROGEL™, an aluminum hydroxy gel adjuvant, (Alhydrogel 1.3%, Alhydrogel 2.0%, or Alhydrogel “85”) obtained from Brenntag Stinnes Logistics can be used.

In addition, MN51 can be combined with an aggregate of an amyloidogenic polypeptide to form a composition that elicits a generic anti-amyloid immune response when administered to a mammal. MN51 (MONTANIDE® Incomplete SEPPIC Adjuvant (ISA) 51) as well as MN720 are available from Seppic (Paris, France). MN51 contains mannide oleate (MONTANIDE® 80, also known as anhydro mannitol octadecenoate) in mineral oil solution (Drakeol 6 VR). MONTANIDE® 80 is a limpid liquid with a maximum acid value of 1, a saponification value of 164-172, a hydroxyl value of 89-100, an iodine value of 67-75, a maximum peroxide value of 2, a heavy metal value less than 20 ppm, a maximum water content of 0.35%, a maximum color value of 9, and a viscosity at 25° C. of about 300 mPas. MONTANIDE® associated with oil (e.g., mineral oil, vegetable oil, squalane, squalene, or esters) is known as MONTANIDE® ISA. Drakeol 6 VR is a pharmaceutical grade mineral oil. Drakeol 6 VR contains no unsaturated or aromatic hydrocarbons, and has an A.P.I. gravity of 36.2-36.8, a specific gravity at 25° C. of 0.834-0.838, a viscosity at 100° F. of 59-61 SSU or 10.0-10.6 centistokes, a refractive index at 25° C. of 1.458-1.463, a better than minimum acid test, is negative for fluorescence at 360 nm, is negative for visible suspended matter, has an ASTM pour test value of 0-15° F., has a minimum ASTM flash point of 295° F., and complies with all RN requirements for light mineral oil and ultraviolet absorption. MN51 contains about 8 to 12 percent anhydro mannitol octadecenoate and about 88 to 92 percent mineral oil.

Other adjuvants include immuno-stimulating complexes (ISCOMs) that can contain such components as cholesterol and saponins. ISCOM matrices can be prepared and conjugated to Cu²⁺ using methods such as those described herein. Adjuvants such as FCA, FIA, MN51, MN720, and Al(OH)₃ are commercially available from companies such as Seppic, Difco Laboratories (Detroit, Mich.), and Superfos Biosector A/S (Vedbeak, Demark).

Other immunostimulatory components include, without limitation, muramyldipeptide (e.g., N-acetylmuramyl-L-alanyl-D-isoglutamine; MDP), monophosphoryl-lipid A (MPL), formyl-methionine containing tripeptides such as N-formyl-Met-Leu-Phe, or a bacterial lipopolysaccarhide. Such compounds are commercially available from Sigma Chemical Co. (St. Louis, Mo.) and RIBI ImmunoChem Research, Inc. (Hamilton, Mont.), for example. Additional immunostimulatory components can include pneumovax (an approved human vaccine), CD40L, or IL-12. In other embodiments, an adjuvant is Complete Freund's Adjuvant or Incomplete Freund's Adjuvant.

This document also provides methods for preparing the compositions provided herein. Such methods can involve suspending an amount of an aggregated polypeptide in a suitable amount of a physiological buffer (e.g., PBS), and then combining the aggregate with a suitable amount of an adjuvant/immunostimulatory compound. The combining step can be achieved by any method, including, for example, stirring, shaking, vortexing, or passing back and forth through a needle attached to a syringe.

It is noted that the composition can be prepared in batch, such that enough unit doses are obtained for multiple injections (e.g., injections into multiple mammals or multiple injections into the same mammal). A “unit dose” of a composition refers to the amount of a composition administered to a mammal at one time. A unit dose of the compositions provided herein can contain any amount of an aggregated polypeptide. For example, a unit dose of a composition can contain between about 0.1 μg and about 1 g (e.g., 1 μg, 10 μg, 15 μg, 25 μg, 30 μg, 50 Ag, 100 μg, 250 μg, 280 μg, 300 μg, 500 μg, 750 μg, 1 mg, 10 mg, 15 mg, 25 mg, 30 mg, 50 mg, 100 mg, 250 mg, 280 mg, 300 mg, 600 mg, 750 mg, or more) of an aggregated polypeptide. For example, a unit dose of a composition can contain between 0.1 μg and 500 μg of an aggregated polypeptide. In some embodiments, the aggregated polypeptide can be suspended or dissolved in a physiological buffer such as, for example, water or phosphate buffered saline (PBS), pH 7.0.

Similarly, a unit dose of a composition can contain any amount of an adjuvant. For example, a unit dose can contain between about 10 μL and about 1 mL (e.g., 10 μL, 25 μL, 50 μL, 100 μL, 250 μL, 500 μL, 750 μL, 800 μL, 900 μL, or 1 mL) of one or more adjuvants. In addition, a unit dose of a composition can contain any amount of an immunostimulatory component. For example, a composition provided herein can contain between about 10 μg and about 1 g (e.g., 10 μg, 15 μg, 25 μg, 30 μg, 50 μg, 100 μg, 250 μg, 280 μg, 300 μg, 500 μg, 750 μg, 1 mg, 10 mg, 15 mg, 25 mg, 30 mg, 50 mg, 100 mg, 250 mg, 280 mg, 300 mg, 500 mg, 750 mg, or more) of an immunostimulatory component.

The compositions provided herein can contain any ratio of adjuvant to aggregated polypeptide. The adjuvant:antigen ratio can be 50:50 (vol:vol), for example. Alternatively, the adjuvant:antigen ratio can be, without limitation, 90:10, 80:20, 70:30, 64:36, 60:40, 55:45, 40:60, 30:70, 20:80, or 90:10.

### Methods of Inducing Generic Anti-Amyloid Humoral Responses

Methods for inducing a generic anti-amyloid humoral response in a mammal (e.g., a mouse, a rat, a cat, a dog, a horse, a cow, a non-human primate such as a cynomolgus monkey, or a human) include administering to a mammal an amount of an aggregate of a heterologous amyloidogenic polypeptide effective for producing the generic anti-amyloid humoral immune response. In some embodiments, a composition described above can be administered to the mammal.

The polypeptides or compositions provided herein can be administered by a number of methods. Administration can be, for example, topical (e.g., transdermal, ophthalmic, or intranasal); pulmonary (e.g., by inhalation or insufflation of powders or aerosols); oral; or parenteral (e.g., by subcutaneous, intrathecal, intraventricular, intramuscular, or intraperitoneal injection, or by intravenous drip). Administration can be rapid (e.g., by injection) or can occur over a period of time (e.g., by slow infusion or administration of slow release formulations).

In some cases, a polypeptide provided herein can be pegylated, acetylated, or both. In some cases, a polypeptide provided herein can be covalently attached to oligomers, such as short, amphiphilic oligomers that enable oral administration or improve the pharmacokinetic or pharmacodynamic profile of the conjugated polypeptide. The oligomers can comprise water soluble polyethylene glycol (PEG) and lipid soluble alkyls (short chain fatty acid polymers). See, for example, International Patent Application Publication No. WO 2004/047871. In some cases, a polypeptide provided herein can be fused to the Fc domain of an immunoglobulin molecule (e.g., an IgGl molecule) such that active transport of the fusion polypeptide across epithelial cell barriers occurs via the Fc receptor. In some cases, a polypeptide can be a cyclic polypeptide.

Any dose can be administered to a mammal. Dosages can vary depending on the relative potency of individual compositions, and can generally be estimated based on data obtained from in vitro and in vivo animal models. Typically, dosage is from about 0.01 μg to about 100 g per kg of body weight, and may be given once or more daily, weekly, or even less often. Following successful administration, it may be desirable to have the subject undergo additional booster administrations to maintain a suitable level of the anti-amyloid response. For example, an additional dosage can be administered 6, 12, 24, 36, 48, 60 or more months after an initial dosage. In some cases, additional dosages can be administered every 6, 12, 18, 24, 30, 36, 42, 48, 54, 60 or more months after an initial dosage. Additional dosages also can be administered as needed.

The anti-amyloid antibody titer of the mammal can be assessed using any method, including, for example, an enzyme-linked immunosorbent assay (ELISA), immunocytochemistry, or Western blotting. For example, plasma or serum from the mammal can be monitored for the ability to detect at least one amyloid (e.g., two or more different amyloids) in vitro, where each tested amyloid is formed by a polypeptide non-homologous with the amyloidogenic polypeptide. In some embodiments, the plasma or serum from the patient can be monitored to detect an amyloid containing Aβ. In other embodiments, the plasma or serum from the patient can be monitored to detect two or more of the following: an amyloid containing Aβ, an amyloid containing an adenovirus shaft fiber peptide (e.g., a polypeptide having the sequence set forth in SEQ ID NO:7), or an amyloid containing a cold shock protein (e.g., a polypeptide having the sequence set forth in SEQ ID NO:2). In some embodiments, the plasma or serum from the mammal is screened for the ability to detect amyloids in situ (e.g., in human or mouse tissue containing Aβ plaques).

In other embodiments, compositions provided herein are administered to a mammal (e.g., a human patient) to alter systemic amyloid deposition in the mammal, including Aβ deposition. Plasma or serum from the mammal can be monitored for the ability to detect fibrillar amyloid beta peptide, serum amyloid A, kappa light chain amyloid and transthyretin in vitro and/or in situ.

In some embodiments, blood drawn from a mammal can be incubated with one or more than one amyloid polypeptide, such as an amyloid polypeptide containing Aβ. The amyloid polypeptides can be heterologous to the amyloidogenic polypeptide used to immunize the mammal. The amyloid polypeptides also can be homologous to the mammal. If antibodies that can recognize the amyloid polypeptides are present in the blood, then the antibodies can bind to the amyloid polypeptides. A secondary antibody can be used to bind to the anti-amyloid antibodies such that the presence or absence of anti-amyloid antibodies bound to amyloid polypeptides can be determined. The secondary antibody can be labeled for detection.

### Articles of Manufacture

Amyloidogenic polypeptide compositions or vaccines described herein can be combined with packaging materials and sold as articles of manufacture or kits. Components and methods for producing articles of manufactures are well known. The articles of manufacture may combine one or more amyloidogenic polypeptide compositions or vaccines described herein. In addition, the articles of manufacture may further include sterile water, pharmaceutical carriers, buffers, antibodies, indicator molecules, and/or other useful reagents for monitoring the immune response of a mammal. Instructions describing how an amyloidogenic polypeptide composition or vaccine is effective for inducing a generic anti-amyloid immune response or altering amyloid deposition can be included in such kits. The amyloidogenic polypeptide composition or vaccine can be provided in a pre-packaged unit dose form in quantities sufficient for a single administration (e.g., for a single human) or for a pre-specified number of humans in, for example, sealed ampoules, capsules, or cartridges.

The invention will be further described in the following examples, which do not limit the scope of the invention described in the claims.

### EXAMPLE

### Generation of a Generic Anti-Amyloid Response by Heterologous Amyloid Polypeptides

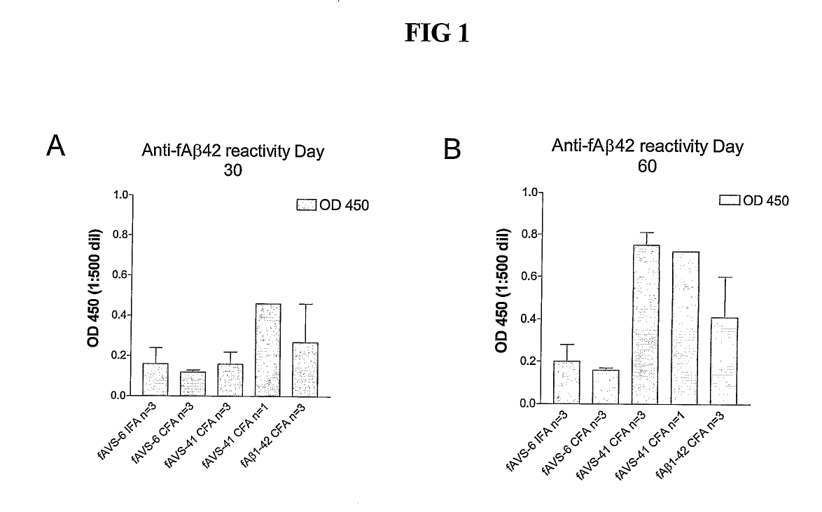

Fibrillar aggregates of adenovirus shaft 6 (fAVS-6, GAITIG, SEQ ID NO:3), adenovirus shaft41 (fAVS-41 PIKTKIGSGIDYNENGAMITKLGSGLSFDNSGAITIGNKND, SEQ ID NO:7) and fAβ1-42 (DAEFRHDSGYEVHHQKLVFFAEDVGSNKGAIIGLMVGGVVIA, SEQ ID NO: 17) were prepared by incubating synthetic polypeptides (2 mg/mL) in phosphate buffered saline (PBS) overnight at 37° C. Mice (B6/SJL F1, 6 weeks old) were subcutaneously injected with the fAVS-6, fAVS-41, and fAβ1-42 polypeptides. Primary injection was on day zero followed by two boosts (days 14 and 46). Each injection (or boost) was 100 μL of a 2 mg/ml fibrillar amyloid preparation stock, emulsified 1:1 in either complete (CFA) or incomplete (IFA) Freund's adjuvant. Sera were collected on day 30 (FIG. 1A) and 60 (FIG. 1B) and antibody titer was tested using a 96 well-ELISA plate coated with fibrillar Aβ1-42 (5 μg/well). The coated plates were blocked then incubated with diluted immune sera (1:500) from immunized mice then secondary antibody (anti-horseradish peroxidase) and developed in peroxidase substrate.

As indicated in FIGS. 1A and 1B, sera (diluted 1:500) from the immunized mice were immunoreactive against fAβ1-42 amyloid. Data shown represent OD at 450 minus the background from normal mouse sera (which gives a background OD 450 of 0.08). Regardless of the fibrillar polypeptide (amyloid) injected, all sera showed reactivity against fAβ1-42 amyloid. The second boost increased the reactivity across the spectra, particularly for fAVS 41. Reactivity of fAVS41 immunized mice sera proved to be equal to or better than sera from the mice injected with fAβ1-42. fAVS 41 showed reactivity when combined with IFA or CFA, indicating that a major response was initiated against fibrillar material itself. These data show that fAVS-6 and AVS-41 induce a generic anti-amyloid response capable of detecting an amyloid formed by a polypeptide that shares no primary sequence homology with the immunogen.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for inducing a generic anti-amyloid immune response in a mammal, said method comprising administering to said mammal an amount of a heterologous amyloidogenic polypeptide effective for producing said generic anti-amyloid immune response, and monitoring plasma or serum from said mammal for the ability to detect a different amyloid in vitro, wherein said different amyloid is formed by a polypeptide non-homologous with said heterologous amyloidogenic polypeptide.

2. The method of claim 1, wherein said plasma or serum is monitored for the ability to detect at least two different amyloids in vitro.

3. The method of claim 1, wherein said non-human amyloidogenic polypeptide is from the shaft sequence of an adenovirus fiber polypeptide.

4. The method of claim 3, wherein said amyloidogenic polypeptide has the sequence set forth in SEQ ID NO:3, SEQ ID NO:4, SEQ ID NO:5, SEQ ID NO:6, or SEQ ID NO:7.

5. The method of claim 1, wherein said amyloidogenic polypeptide is selected from the group consisting of the chorion class A polypeptide (SEQ ID NO:8) and the chorion class B polypeptide (SEQ ID NO: 9).

6. The method of claim 1, wherein said amyloidogenic polypeptide is a N-terminal fragment of a bacterial cold shock protein.

7. The method of claim 6, wherein said amyloidogenic polypeptide is the polypeptide set forth in SEQ ID NO:1 or the polypeptide set forth in SEQ ID NO:2.

8. The method of claim 1, wherein said amyloidogenic polypeptide is a curlin protein having the amino acid sequence set forth in SEQ ID NO: 12.

9. The method of claim 8, wherein said amyloidogenic polypeptide is a fragment of the polypeptide set forth in SEQ ID NO: 12.

10. The method of claim 1, wherein said amyloidogenic polypeptide is a curlin related protein.

11. The method of claim 10, wherein said curlin related protein is the AgfA protein having the amino acid sequence set forth in SEQ ID NO:13.

12. The method of claim 1, wherein said amyloidogenic polypeptide is a fragment of Sup35 or Ure2p from Saccyromyces.

13. The method of claim 1, wherein said amyloidogenic polypeptide is antifreeze polypeptide-3 having the amino acid sequence set forth in SEQ ID NO: 15 or a related polypeptide.

14. The method of claim 1, wherein said mammal is a human.

15. The method of claim 1, wherein said amyloidogenic polypeptide is a fragment of HET-s protein from Podospora anserina.

16. The method of claim 1, wherein said amyloidogenic polypeptide is a fungal hydrophobin.

17. The method of claim 1, wherein said amyloidogenic polypeptide is a chaplin from Streptomycetes spp.

18. The method of claim 1, wherein said amyloidogenic polypeptide is the monnelin chain A polypeptide of SEQ ID NO: 10 or the monellin chain B polypeptide of SEQ ID NO: 11.

19. The method of claim 1, wherein said amyloidogenic polypeptide is FlgB, FlgC, FlgG or FliE.

20. The method of claim 1, wherein said amyloidogenic polypeptide is a fragment of FlgB, FlgC, FlgG or FliE.

21. The method of claim 1, wherein said amyloidogenic polypeptide is an all D-enantiomer.

22. The method of claim 1, wherein said amyloidogenic polypeptide is a mixed D and L enantiomer.

23. The method of claim 1, wherein the amyloidogenic polypeptide is Boc-β-Ala-mABA-Ome or Boc-γ-Abu-mABA-Ome.

24. The method of claim 1, further comprising monitoring plasma or serum from said mammal for the ability to detect amyloid deposits in human tissue.

25. A composition comprising a fibrillar aggregate of a nonhuman amyloidogenic polypeptide and an adjuvant.

26. The composition of claim 25, wherein said adjuvant is alum.

27. The composition of claim 25, wherein said non-human amyloidogenic polypeptide has the sequence set forth in SEQ ID NO:3, SEQ ID NO:4, SEQ ID NO:5, SEQ ID NO:6, or SEQ ID NO:7.

28. The composition of claim 25, wherein said non-human amyloidogenic polypeptide is selected from the group consisting of the chorion class A polypeptide (SEQ ID NO:8) and the chorion class B polypeptide (SEQ ID NO: 9).

29. The composition of claim 25, wherein said non-human amyloidogenic polypeptide is a N-terminal fragment of a bacterial cold shock protein.

30. The composition of claim 25, wherein said non-human amyloidogenic polypeptide is the polypeptide set forth in SEQ ID NO:1, SEQ ID NO:2, SEQ ID NO:10, or SEQ ID NO:11.

31. The composition of claim 25, wherein said non-human amyloidogenic polypeptide is a curlin protein having the amino acid sequence set forth in SEQ ID NO: 12 or a fragment of the polypeptide set forth in SEQ ID NO: 12.

32. The composition of claim 25, wherein said non-human amyloidogenic polypeptide is the AgfA protein having the amino acid sequence set forth in SEQ ID NO:13.

33. The composition of claim 25, wherein said non-human amyloidogenic polypeptide is a fragment of Sup35 or Ure2p from Saccyromyces.

34. The composition of claim 25, wherein said non-human amyloidogenic polypeptide is antifreeze polypeptide-3 having the amino acid sequence set forth in SEQ ID NO: 15 or a related polypeptide.

35. The composition of claim 25, wherein said non-human amyloidogenic polypeptide is a fragment of HET-s protein from Podospora anserina.

36. The composition of claim 25, wherein said non-human amyloidogenic polypeptide is a fungal hydrophobin.

37. The composition of claim 25, wherein said non-human amyloidogenic polypeptide is a chaplin from Streptomycetes spp.

38. The composition of claim 25, wherein said non-human amyloidogenic polypeptide is FlgB, FlgC, FlgG or FliE or a fragment of FlgB, FlgC, FlgG or FliE.

39. The composition of claim 25, wherein said non-human amyloidogenic polypeptide is an all D-enantiomer.

40. The composition of claim 25, wherein said non-human amyloidogenic polypeptide is a mixed D and L enantiomer.

41. The composition of claim 25, wherein said non-Human amyloidogenic polypeptide is Boc-β-Ala-mABA-Ome or Boc-γ-Abu-mABA-Ome.

42. The composition of claim 25, wherein said aggregate is an amyloid,

43. The composition of claim 25, wherein said aggregate is a soluble oligomer, annular pore, or protofibril.

44. A method for altering Aβ deposition in a patient, said method comprising administering a composition to said patient, said composition comprising a non-human amyloidogenic polypeptide and an adjuvant.

45. The method of claim 44, said method farther comprising monitoring plasma or serum from said patient for the ability to detect fibrillar amyloid beta polypeptide in vitro or in situ.
